(19) United States
(12) Patent Application Publication
(10) Pub. No.: US 2016/0073883 A1
(43) Pub. Date: Mar. 17, 2016

(54) DEVICE AND METHOD FOR ACQUIRING BIOLOGICAL INFORMATION BY MEANS OF AN INTRACORPOREAL CURRENT

(71) Applicant: INSIDE SECURE, Meyreuil (FR)

(72) Inventors: Bruno CHARRAT, AIX EN PROVENCE (FR); Georges GAGNEROT, MARSEILLE (FR)

(21) Appl. No.: 14/784,785

(22) PCT Filed: Mar. 28, 2014
(86) PCT No.: PCT/FR2014/050745

§ 371 Date: Oct. 15, 2015

(30) Foreign Application Priority Data
Apr. 15, 2013 (FR) 13 53384

(51) Int. Cl. A61B 5/00; A61B 5/021

(52) U.S. Cl. CPC A61B 5/0028; A61B 5/021; A61B 5/7278; A61B 5/7228

(57) ABSTRACT

A receiving device with intracorporeal current having a device for collecting, by capacitive coupling, an AC signal depending on a current that has passed through all or part of the body of a subject, a device for extracting data from the AC signal collected, and a device for extracting from the AC signal a biological signal generated by the body of the subject and modulating the amplitude of the AC signal.

The present invention relates to a method and a device for acquiring a biological signal. The present invention also relates to IBAN (“Intra Body Area Network”) data transmission techniques of the type described in the European patent EP 0 824 799 and in the document “Personal Area Networks (PAN)—Near-Field Intra-Body Communication”, by Thomas Guthrie Zimmerman, Massachusetts Institute of Technology, September 1995.

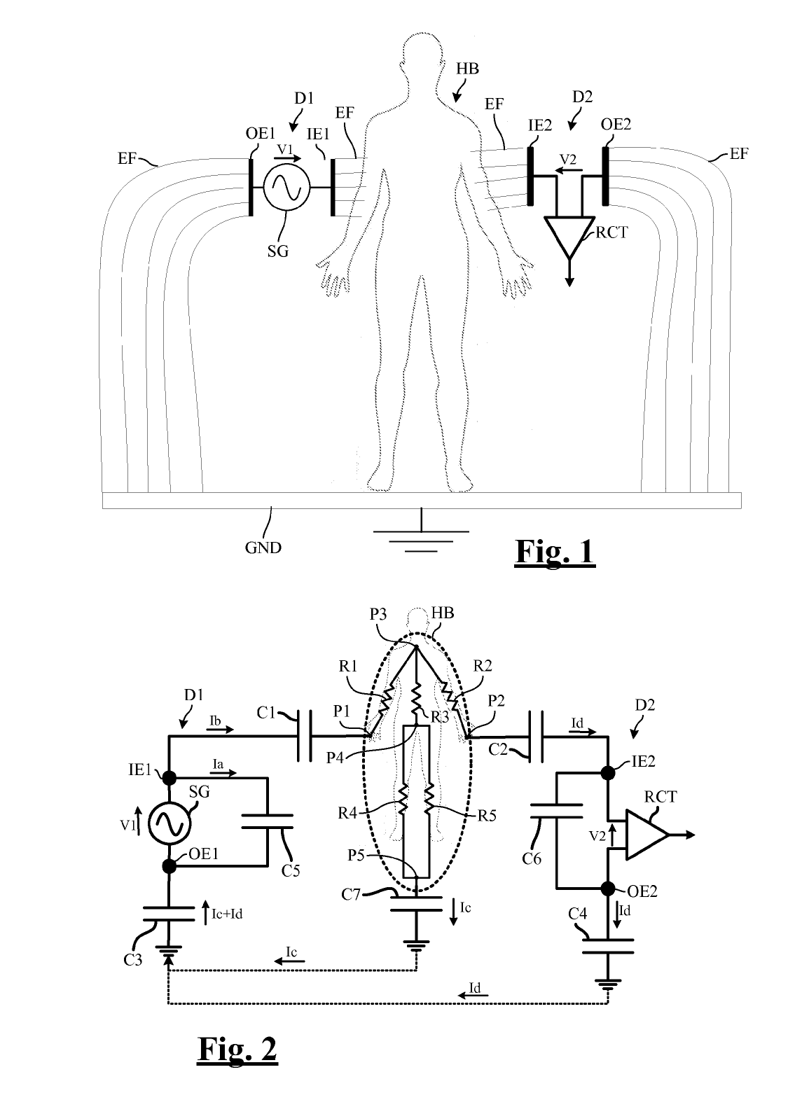

FIG. 1 schematically shows an IBAN system comprising a transmitter D1, a receiver D2, and the body HB of a subject as transmission medium. The transmitter D1 comprises an external electrode OE1, or environment electrode, an internal electrode IE1, or body electrode, and a voltage generator SG coupled to the two electrodes. The receiver D2 also comprises an external electrode OE2 and an internal electrode IE2.

The generator SG of the transmitter D1 creates an oscillating potential V1 between the electrodes OE1, IE1. An electric field EF forms between the internal electrode IE1 and the body HB of the subject, and between the external electrode OE1 and the environment. The body HB is considered to be a large capacitor plate which can be charged and discharged by the transmitter D1. The environment is schematically represented by the floor, and has a reference potential considered as forming the ground GND of the IBAN system. The electric charge applied to the body of the subject gives it a potential different from that of the environment, which causes the appearance of an electric field EF between the body and the environment and between the body and the receiver D2. A voltage V2 appears on the electrode IE2 of the receiver D2. A receiver circuit RCT measures the voltage V2, relative to the potential of the external electrode OE2.

FIG. 2 is a representation of the IBAN network of FIG. 1 in the form of a capacitive and resistive electric network. A capacitor C1 represents the capacitive coupling between the internal electrode IE1 of the device D1 and a zone of the body the most proximate to this electrode, schematically represented by a point PH1. A capacitor C2 represents the capacitive coupling between the internal electrode IE2 of the device D2 and a zone of the body the most proximate to this electrode, schematically represented by a point P2. A capacitor C3 represents the capacitive coupling between the external electrode OE1 of the device D1 and the environment. A capacitor C4 represents the capacitive coupling between the external electrode OE2 of the device D2 and the environment. A capacitor C5 represents the capacitive coupling between the electrodes OE1 and IE1. A capacitor C6 represents the capacitive coupling between the electrodes OE2 and IE2, and a capacitor C7 represents the capacitive coupling between the feet and the environment. Other coupling capacitors featured in the model of the Massachusetts Institute of Technology are not represented here for the sake of simplicity.

Furthermore, the body is considered to be a purely resistive node schematically represented by resistors R1, R2, R3, R4, R5. The resistors R1 and R2 are in series and pass via a fictitious midpoint P3. They illustrate the total electrical resistor of the body between the points P1 and P2. Assuming for example that the user capacitively couples the devices D1 and D2 by means of its right and left hands, the resistor R1 is the resistor of the right arm and right shoulder, and the resistor R2 is the resistor of the left shoulder and left arm, the midpoint P3 being located between the two shoulders. The resistor R3 links the point P3 to a fictitious point P4 in the vicinity of the pelvis and represents the resistor of the thorax. The resistors R4 and R5 are in parallel and each link the point P4 to a fictitious point P5 coupled to the environment by the capacitor C7, and represent the series resistors of the left and right legs.

When the voltage V1 is applied to the electrodes IE1, OE1, a current is transmitted by the voltage generator SG. A first fraction Ia of this current passes through the capacitor C5 to reach the external electrode OE1, and a second fraction Ib of this current is injected into the body through the capacitor C1, to form an intracorporeal current. A fraction IC of the current Ib passes through the resistor R1, the resistor R3 of the thorax and the resistors R4, R5 of the legs, then the capacitor C7, to then join the external electrode OE1 of the device D1 by passing through the environment and the capacitor C3, the environment being represented by dotted lines. Another fraction Id of the current Ib passes through the resistors R1, R2 and the capacitor C1 to reach the internal electrode IE2 of the device D2, then passes through the device D2 and joins the external electrode OE1 of the device D1 by passing through the environment and the capacitor C3, as also represented by dotted lines. The resistances R3+R4 or R3+R5 can be much higher than the resistance R2, and the current Ic much lower than the current Id. The intracorporeal current Id generates the voltage V2 at the terminals of the electrodes IE2, CF2, and the latter is measured by the receiver circuit RCT.

To transfer data from the device D1 to the device D2, the amplitude of the voltage V1 is modulated by a data carrier signal. The amplitude modulation is reflected in the current Id and in the voltage V2. The device D2 demodulates the current Id or the voltage V2 and extracts the data signal therefrom.

The current Id is very low, as is the voltage V2, which is generally of the order of one millivolt to a few millivolts. Thanks to progress made in the field of microelectronics, integrated circuits on semiconductor chip capable of detecting a very low AC signal and of extracting a data carrier modulation signal from it are today produced to implement IBAN applications. As an example, the MCP2030 and MCP2035 “Analog Front-End Device for BodyCom Applications” integrated circuits marketed by Microchip are specifically designed for IBAN applications.

An IBAN network enables devices proximate to the body to exchange data. One well-known method consists in particular of using an IBAN network to convey a biological parameter measured by means of a sensor, for example a heartbeat sensor, to an information collecting device. In this case, the heart sensor is equipped with a transmitter D1 and the collecting device is equipped with a receiver D2. An IBAN data link is established between the sensor and the collecting device. The latter is equipped with means for storing, analyzing and/or displaying the heartbeat, or for transmitting the latter to a remote device.

Using the heart signal as a means of identifying a person is also well-known. In particular, the Toronto-based company Bionym has developed a product called “HeartID”, comprising biometric identification means based on the analysis of the heart signal. Such a method is implemented by means of a dedicated heart signal sensor, in the form of a personal computer peripheral device.

The present invention is based on the discovery of the fact that an intracorporeal current implemented for an IBAN data transmission can be used to capture biological information.

The present invention relates more particularly to a receiving device with intracorporeal current comprising means for collecting, by capacitive coupling, an AC signal depending on a current that has passed through all or part of the body of a subject, and means for extracting data from the AC signal collected, the device further comprising means for extracting from the AC signal a biological signal generated by the body of the subject and modulating the amplitude of the AC signal.

According to one embodiment, the device is configured to extract or extrapolate from the biological signal at least one biological parameter or one item of biological information.

According to one embodiment, the biological parameter is a parameter involved in transforming the heart signal into a biological signal.

According to one embodiment, the biological parameter consists of the variations in the subject's blood pressure.

According to one embodiment, the biological parameter is the subject's heartbeat.

According to one embodiment, the biological information comprises at least one variation in the biological signal at a given time in the cardiac cycle.

According to one embodiment, the device is configured to develop a biometric identification datum of the subject from one or more biological parameters and/or one or more items of biological information.

According to one embodiment, the device comprises means for transmitting data by applying to the body of the subject, by capacitive coupling, an AC signal modulated by a data signal.

Some embodiments of the present invention also relate to a system with intracorporeal current comprising: a transmitting device comprising means for applying to the body of a subject, by capacitive coupling, a first AC signal, and means for transmitting data via the first AC signal, and a receiving device according to the present invention, to collect, by capacitive coupling, a second AC signal, and to extract from the second AC signal data sent by the transmitting device, wherein the receiving device is configured to, during an initialization phase, exchange data with the transmitting device, and during an acquisition phase, extract the biological signal from the second AC signal.

According to one embodiment, at least one of the two devices is arranged in a portable object.

Some embodiments of the present invention also relate to a method of acquiring a biological signal generated by the body of a subject, comprising the steps of: applying to the body of the subject a first electric AC signal, by means of a transmitting device comprising means for applying the first AC signal to the body of the subject by capacitive coupling, and means for transmitting data via the first AC signal; collecting a second AC signal depending on a current that has passed through all or part of the body of the subject, by means of a receiving device comprising means for collecting, by capacitive coupling, the second AC signal; and means for extracting data from the AC signal collected, and extracting the biological signal from the second AC signal, as signal modulating the amplitude of the second AC signal.

According to one embodiment, the method comprises the steps of: during an initialization phase, exchanging data with the transmitting device, by means of the receiving device, and during an acquisition phase, extracting the biological signal from the second AC signal, by means of the receiving device, as signal modulating the amplitude of the second AC signal.

According to one embodiment, the method comprises a step of extracting or extrapolating from the biological signal at least one biological parameter or one item of biological information.

According to one embodiment, the biological parameter is a parameter involved in transforming the heart signal into a biological signal.

According to one embodiment, the biological parameter consists of the variations in the subject's blood pressure or the subject's heartbeat.

Some embodiments of the method of acquiring the biological signal and of devices according to the present invention will be described below in relation with, but not limited to, the accompanying figures, in which:

FIG. 1 described above schematically represents an IBAN network,

FIG. 2 described above is the wiring diagram of the IBAN network in FIG. 1,

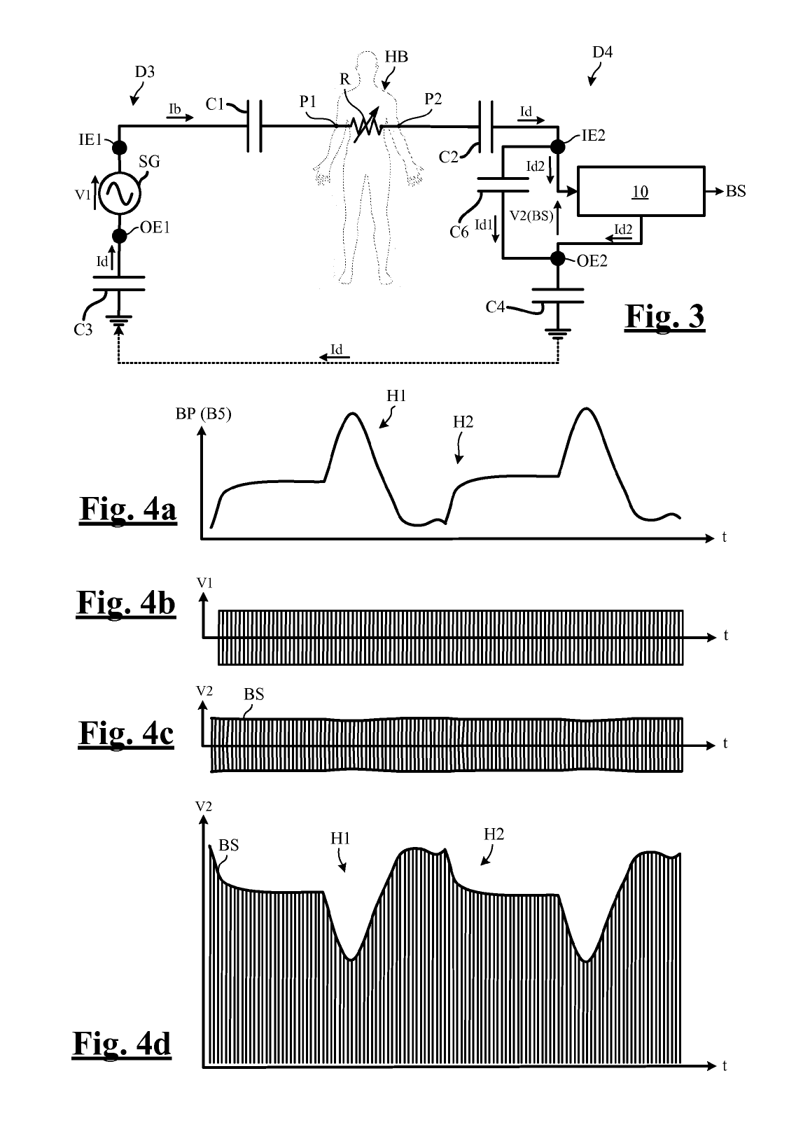

FIG. 3 is the wiring diagram of one embodiment of an IBAN system according to the present invention,

FIG. 4a represents a curve of the blood pressure variations according to the cardiac cycle,

FIG. 4b represents an AC voltage applied at a point of the body of a subject,

FIG. 4c represents an AC voltage collected at another point of the body of the subject,

FIG. 4d is an expanded view of a biological signal present in the AC voltage in FIG. 4c,

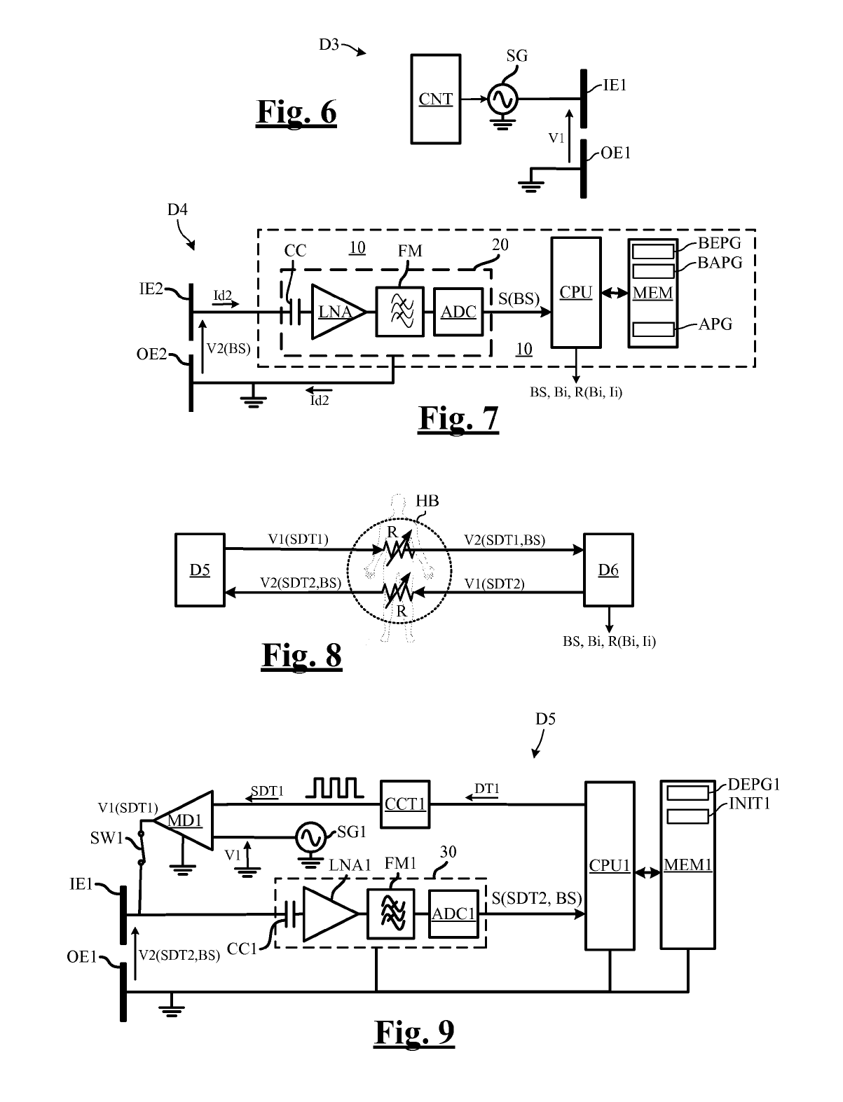

FIGS. 6 and 7 represent some embodiments of a transmitter and of a receiver of the device in FIG. 3,

FIG. 8 represents another embodiment of an IBAN system according to the present invention,

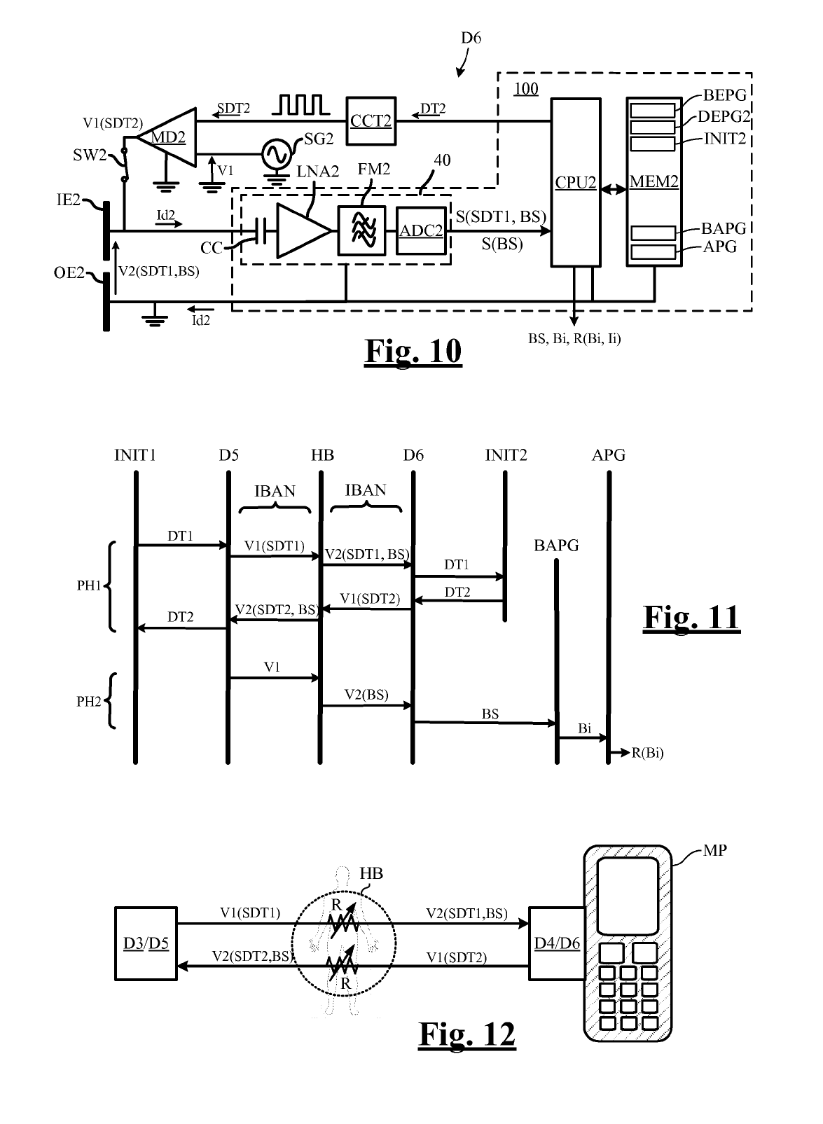

FIGS. 9 and 10 represent some embodiments of a transmitter and of a receiver of the system in FIG. 7,

FIG. 11 is a timing diagram showing the operation of the IBAN system in FIG. 8, and

FIG. 12 shows an application of an IBAN system according to the present invention.

FIG. 3 is a simplified wiring diagram of one embodiment of an IBAN system according to the present invention. The system comprises a transmitting device D3 and a receiving device D4. The transmitter D3 comprises an internal electrode IE1, an external electrode OE1, and a voltage generator SG. The generator SG applies to the electrodes IE1, OE1 an AC voltage V1 the oscillation frequency Fc of which may be preferably between 100 KHz and 20 MHz, for example the standardized frequency of 13.56 MHz used in NFC communications (“Near Field Communication”).

The receiver D4 comprises an internal electrode IE2, an external electrode OE2 and a receiver circuit 10 according to the present invention. The receiver circuit 10 comprises an input coupled to the electrode IE2 and a reference potential terminal coupled to the electrode OE2, and is configured to extract a biological signal BS from a voltage V2 appearing between the electrodes IE2 and OE2 when the voltage V1 is transmitted by the transmitter D3. The surface area of the electrodes may vary from a few square millimeters to a few square centimeters, depending on the intended application and the conditions of implementation of the system. In some embodiments, these electrodes may be associated with antenna coils to form resonant circuits. In other embodiments, these electrodes may be replaced with antennas and generally speaking with any means enabling an electric field to be emitted or sensed.

It is assumed here that the electrode IE1 is capacitively coupled to a point P1 of the body HB of a subject, and that the electrode IE2 is capacitively coupled to a point P2 of the body. The points P1, P2 are fictitious and model zones of inductive coupling between each of the electrodes IE1, IE2 and the body of the subject. Under the action of the electric field generated by the voltage V1, a conducting loop passing through the body of the subject is created. As described above in connection with FIG. 2, this conducting loop may comprise:
a coupling capacitor C1 between the electrode IE1 and the point P1,
a resistor R between the points P1 and P2, representing the electrical resistor of the body (equivalent to the sum of resistances R1 and R2 in FIG. 2),
a capacitor C2 between the point P2 and the electrode IE2,
a capacitor C6 between the electrode IE2 and the electrode OE2,
a capacitor C4 between the electrode OE2 and the environment,
a conducting path passing through the environment, schematically represented by dotted lines, and
a capacitor C3 between the environment and the electrode OE1.

The environment is for example the floor, if the subject is standing without touching any objects located in its environment, or any element of the environment offering a conducting path between the external electrodes OE1, OE2. Other conducting paths or “leak paths” passing through the body or otherwise, such as the conducting paths passing through the legs and the capacitors C5 and C7 shown on FIG. 2, have not been represented on FIG. 3 for the sake of simplicity.

Under the action of the voltage V1 and of the electrostatic field that the latter generates around the subject, relative to the electric potential of the environment, a current Ib is injected at the point P1 of the body. The electrode IE2 then collects a current Id representing a fraction of the current Ib, due to current leakages in other conducting loops. The current Id varies according to the resistor R of the body and generates the voltage V2 between the electrodes IE2 and OE2 of the receiver D4. A portion Id1 of the current Id passes through the capacitor C6 and a portion Id2 of the current Id passes through the receiver circuit 10. The current Id then joins the electrode OE1 by passing through the capacitor C4, the environment (path represented by dotted lines) and the capacitor C3.

According to the findings on which the invention is based, the current Id collected by the electrode IE2 has an amplitude modulation linked to the variations in the resistor R of the body between the points P1 and P2, and these resistance variations depend on the variations in the subject's blood pressure. On the diagram in FIG. 3, the resistor R of the body between the points P1 and P2 is thus represented as a variable resistor of which the value varies with the blood pressure. This gives rise to a corresponding modulation of the current Id2 and of the voltage V2.

Some embodiments of the invention are based on this technical effect of biological origin, that is schematically shown on FIGS. 4a to 4b. FIG. 4a is a curve representing the variations in the subject's blood pressure BP depending on its cardiac cycle. The curve BP has a peak H1 during the systole phase followed by a dip H2 during the diastole phase, and the peak H1 and the dip H2 as represented may have various shapes depending on the subject. FIG. 4b shows the shape of the AC voltage V1 generated by the device IE1. It is assumed here that the voltage V1 is not amplitude modulated, and thus has a constant amplitude. FIG. 4c shows the shape of the AC voltage V2 detected by the circuit 10 on the electrode IE2 of the receiver D4. The envelope of the signal V2 has a low-value amplitude modulation, generally of the order of a few microvolts, which can be drowned in background noise. This background noise, not represented on the figure, may be random or synchronous and it may particularly be generated by electrical equipment of 50 or 60 Hz located in the environment of the subject. Once the noise is removed, the signal V2 appears as the result of an amplitude modulation of the signal V1 by a biological signal BS, which thus forms an envelope signal of the signal V2. The signal BS reflects the variations in the current Id according to the variations in the resistor R of the body, itself varying with the blood pressure.

FIG. 4d is an expanded view of the variations in the amplitude of the voltage V2 in the vicinity of its maximum value. The biological signal BS is generated by the body of the subject and has variations that are the opposite of those of the blood pressure BP, which indicates that the resistor R of the body decreases when the blood pressure increases.

Thus, the receiver circuit 10 of the device D4 is configured to extract the signal BS from the signal V2, by using any appropriate envelope extracting technique, including the removal of the carrier Fc and the elimination of the random or synchronous noise which may mask the signal BS.

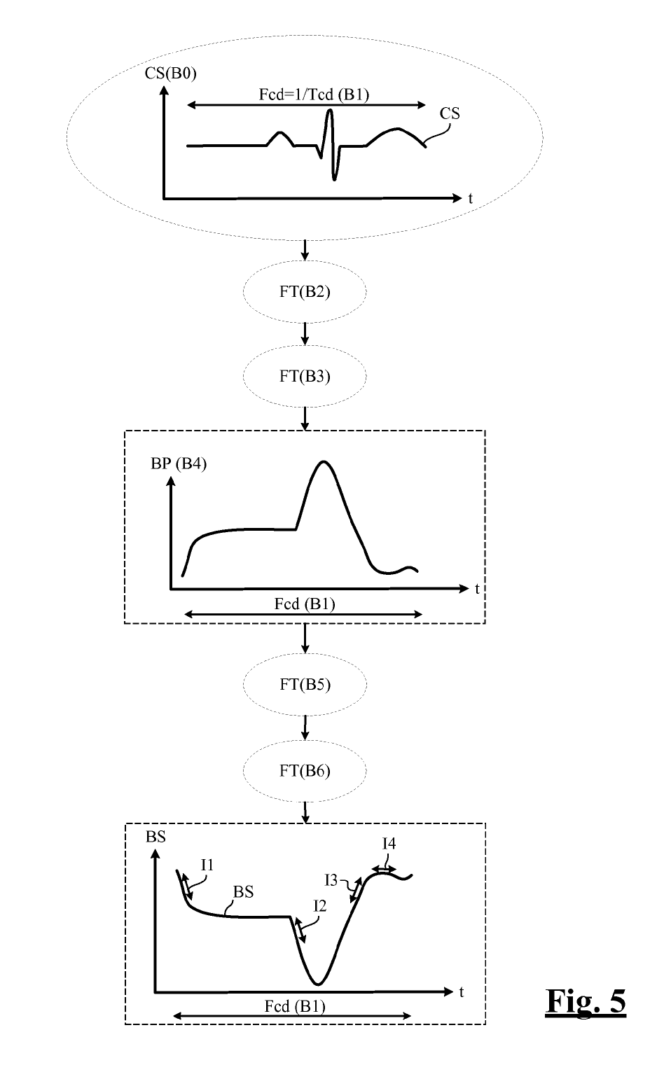

FIG. 5 schematically represents, without limitation, a possible example of the relationship between the biological signal BS, the variations in blood pressure BP, and various biological parameters Bi (B1, B2, B3, etc.) which contribute to the existence and the shape of the signal BS. At the origin of the signal BS two basic parameters B0 and B1 can be distinguished. B0 is the heart signal CS or electrocardiogram) and B1 is the heart rate Fcd, equal to the opposite of the heart period Tcd.

An experimental and non-limitative modeling of the biological system is proposed here that represents the body, in which the body is considered to comprise a set of “transfer functions” FT(Bi) (or transformation functions) each depending on a biological parameter Bi and which, using the two basic biological parameters B0 and B1, lead to obtaining the biological signal BS measurable with the above-mentioned extracting technique. It will be noted that what is explained here in connection with FIG. 5 relates only to certain aspects of certain embodiments of the invention and relies on assumptions requiring subsequent research and development work for them to be exploited for application purposes. The present invention thus opens up a vast field of exploration and application possibilities requiring additional studies.

Only four transformation functions FT(B2), FT(B3), FT(B5), FT(B6) are represented for the sake of simplicity. The functions FT(B2), FT(B3)) are cumulative and transform the heart signal CS into blood pressure BP variations. The blood pressure variations BP are themselves considered an intermediate biological parameter B5. The functions FT(B5), FT(B6) are also cumulative and transform the blood pressure variations BP into a measurable biological signal BS. It is considered that the biological parameters Bi can be extracted or extrapolated from the signal BS. In some cases, extracting or extrapolating a biological parameter Bi may require knowing all or part of the other biological parameters.

The biological parameter B2 represents for example the shape of the heart, its tonus, the quality of the heart muscles, and indirectly the age of the subject. Generally speaking, the function FT(B2) represents for example the ability of the heart to transform the heart signal into blood pressure variations. The parameter B3 represents for example the activity of the subject at the time the biological signal BS is measured, and the function FT(B3) represents for example the influence of the subject's activity on the variations in its blood pressure. For example, the blood pressure signal BP may vary in a different manner depending on whether the subject is resting, hopping on the right leg or the left leg, walking, running, etc. The parameter B5 represents for example the irrigation of the body tissues in the region through which the current Ib passes, and the function FT(B5) represents for example a function for transforming the blood pressure variations into variations in the resistivity of the tissue in the region passed through by the current Ib, which may vary depending on whether or not the tissue is properly irrigated. The parameter B6 represents for example the state of the subject's hydration, and the function FT(B6) represents for example a function for transforming the blood pressure variations into variations in the resistivity of the tissue in the region passed through by the current Ib, which may vary depending on whether or not the tissue is properly hydrated.

Knowing the signal BS may enable certain biological parameters to be extracted or extrapolated, in a simple or more complex manner depending on the parameter sought. For example, knowing the signal BS may first of all enable the heart rate Fcd to be determined, which is also the frequency of the signal BS. Furthermore, assuming that the transformation functions FT(25), FT(B6) are not active, the signal BS enables the blood pressure signal BP to be found, one being the opposite of the other. In a more complex manner, the functions FT(B5), FT(B6) may be calibrated by measuring the blood pressure variations BP by means of an appropriate instrument, while measuring the signal BS, and by correlation between the shape of the signal BS and the blood pressure variations measured. Similarly, measuring the heart signal CS by means of an appropriate instrument may enable a relationship to be established between the exact shape of the heart signal CS and that of the signal BS, or between the exact shape of the heart signal CS and the shape of the curve of blood pressure variations BP, which can then enable the heart signal CS to be extrapolated from the biological signal BS.

Knowing the signal BS may also enable this signal to be extracted or extrapolated from the biological information Ii, which is directly or indirectly representative of biological parameters Bi. For example, the slope of variation of the signal BS at a first point of the curve of the signal BS, or local derivative of the signal BS, can be a first item of biological information I1, the local derivative of the signal BS at a second point of the curve of the signal BS can be a second item of biological information I2, the derivative at a third point of the curve a third item of biological information I3, the derivative at a fourth point of the curve a fourth item of biological information I4, and so on. These various measurement points of the derivative can be easily located on the curve of the signal BS by referring to the cardiac cycle, which is also the cycle of the signal BS.

The information Ii extracted in this way from the signal BS is representative of the blood pressure variations BP, as seen on FIG. 5, but the variations of the items of information I1 to I4 over time may themselves be other items of biological information representative of a change in the biological parameters B2, B3, B5, B6. In other words, for the same subject, the same heart signal may result, at different times, in different variations in the blood pressure depending on the state of the subject's heart (parameter B2) or on the activity of the subject (parameter B3), and a same variation in the blood pressure may result, at different times, in different variations in the conductivity of the tissue depending on the irrigation of the tissue (parameter B5) or on the hydration of the tissue (parameter B6).

FIGS. 6 and 7 respectively represent one embodiment of the transmitter D3 and of the receiver D4. The transmitter D3 comprises a control circuit CNT activating and deactivating the generator SG and optionally setting the amplitude of the signal V1. The receiver circuit 10 of the device D4 comprises an acquisition chain 20 for acquiring the biological signal BS, a processor CPU and a program memory MEM. The memory MEM comprises the operating system of the processor CPU and a program BEPG for extracting the signal BS. The acquisition chain 20 comprises a decoupling capacitor CC, a low noise amplifier LNA, a band-pass filter FM and an analog-digital converter ADC the output of which is coupled to a port of the CPU. The amplifier LNA is coupled to the electrode IE2 through the decoupling capacitor CC. The output of the amplifier is coupled to the input of the converter ADC through the band-pass filter FM. The amplifier LNA may be a voltage amplifier and amplify the voltage V2, or a current amplifier and amplify the fraction Id2 of the current Id that passes through it, the signal at output of the acquisition chain being in any cases a signal S(BS) that is the image of the current Id and the image of the voltage V2.

The filter FM has a bandwidth centered on the carrier frequency Fc to eliminate the noises situated outside the IBAN frequency band, such as the noise at 50 Hz or 60 Hz generated by electric appliances and the random noise, and to only allow the carrier Fc and the biological signal BS to pass. For example, if the frequency Fc is 10 MHz, the filter FM is centered on 10 Mhz with a bandwidth ranging from 9 to 11 Mhz, to supply the converter ADC with a “clean” signal S(BS) having a central band at 10 MHz and side bands carrying the biological signal BS.

The program BEPG executed by the processor CPU then carries out the demodulation and the low-pass filtering of the signal S(BS), the demodulation making it possible to remove the carrier Fc and the filtering making it possible to extract the biological signal BS from the demodulated signal. In one alternative embodiment, these demodulation and low-pass filtering steps can be performed with an analog demodulator and a low-pass filter arranged between the filter FM and the converter ADC.

According to one embodiment, the memory MEM further comprises a biological analysis program BAPG enabling the processor CPU to extract or extrapolate from the biological signal BS a biological parameter Bi or an item of biological information Ii of the type previously described, or any other parameter or biological information susceptible of being later brought to light, which the processor may possibly supply at an output port.

The biological analysis program BAPG is for example configured to extract the heart rate Fcd from the signal BS, by measuring the frequency of this signal. The program BAPG may also use a database stored in the memory MEM, generated during a calibration phase, or an extrapolation function developed by experiments, to reconstitute the heart signal CS of the subject from the signal BS. The program BAPG may also search in the biological signal BS for one of the other biological parameters Bi described above.

According to one embodiment, the memory MEM also comprises a biological application program APG that uses the biological signal BS, the biological parameter Bi or the biological information Ii supplied by the program BAPG, to obtain a result R(Bi, Ii) that the processor CPU may possibly supply at an output port. The application program APG may for example be
a program monitoring the health of the subject, which compares the curve of the signal BS at a given time with a curve of the signal BS stored at a previous time, or which compares a biological parameter Bi or an item of biological information Ii supplied by the program BAPG, with a biological parameter Bi or an item of biological information Ii previously measured, or a reference biological parameter or item of information relating to the subject's state of health. The result R may then consist of an item of information about the subject's state of health;
a sleeping detection program, which monitors the signal BS. For this purpose, the program may use a combination of biological parameters Bi and of biological information Ii which can be extracted from the signal BS, for example the heart rate Fcd and local derivatives of the signal BS. The result R(Bi, Ii) may then consist of an alert, which can be sent to an external device so as to generate a visual or sound alarm;
a program executing steps of biometric identification of the subject. In this case, the program APG generates from the signal BS a biometric identification datum of the subject. This identification datum is for example a template which defines the general shape of the signal BS, considered to be unique and specific to the subject, like the variations in the heart signal CS, already used in prior art as biometric identification signal. The program APG may use any known method to define this template, for example using a set of local derivatives of the signal BS enabling the template to be defined. The program APG then compares this signature with a signature previously stored, and supplies a positive or negative result (success or failure).
an encryption program using as encryption key one or more items of biological information Ii obtained from the signal BS, for example one or more local derivatives of the signal BS. The result R may then be the result of the transformation of a datum or of a message by the encryption function.

FIG. 8 schematically represents another embodiment of an IBAN system according to the present invention. The transmitting device D3 is replaced with a transmit-receive device D5 and the receiving device D4 is replaced with a transmit-receive device D6. The devices D5, D6 are configured to exchange data via the body HB, by means of an IBAN signal, in a conventional manner per se. The device D6 is also configured to extract the biological signal BS from the IBAN signal transmitted by the device D5. The system preferably works in two phases PH1 and PH2, phase PH1 being an initialization phase and PH2 an acquisition phase of acquiring the biological signal BS by the device D6.

During the phase PH1, the two devices exchange data and define the beginning of the phase PH2. The device D5 is an “initiator” and the device D6 is a “target”. The device D5 goes into transmitting mode and transmits an AC voltage V1(SDT1) of frequency Fc which is amplitude modulated by a data signal SDT1. The signal SDT1 is preferably an AC signal of a frequency lower than that of the carrier Fc, for example a signal of a few hundred kilohertz if the carrier Fc is of the order of a few megahertz. The device D6, by default in receiving mode, receives an AC voltage V2(SDT1, BS) which is modulated by the data signal SDT1. The device D6 extracts the data signal SDT1 from the voltage V2, then extracts the data DT1 included in the signal SDT1.

As the voltage V2 depends on an IBAN current that has passed through the body HB of the subject, the resistor R of which varies with the blood pressure, it is also and necessarily amplitude modulated by the biological signal BS. However, the device D6 preferably does not extract the signal BS from the voltage V2 during the phase PH1. As the signal BS is a low frequency signal, its extraction would considerably slow down the execution of the phase PH1.

When the device D5 has transmitted the data DT1, it stops supplying the voltage V1, switches to receiving mode and waits for a response from the device D6. After extracting the data DT1, the device D6 in turn transmits an AC voltage V1(SDT2), of frequency Fc, amplitude modulated by a data signal SDT2. The device D5 receives an AC voltage V2(SDT2, BS) which is modulated by the data signal SDT2. The device D5 extracts the data signal SDT2 from the voltage V2, then extracts the data DT2 from the data signal SDT2. It will be noted that the voltage V2 is also amplitude modulated by the biological signal BS, but that the device D5 does not comprise here any means of extracting this signal.

The devices D5 and D6 exchange data DT1, DT2 until the beginning of the acquisition phase PH2. During the phase PH2, the device D5 switches to transmitting mode and transmits the AC voltage V1 without modulating its amplitude. The device D6 switches to receiving mode and receives a voltage V2(BS) amplitude modulated by the biological signal BS, from which it extracts the signal BS.

FIG. 9 shows an example of an embodiment of the device D5. The latter comprises a processor CPU1 coupled to a memory MEM1, means for transmitting data and means for receiving data. The memory MEM1 comprises a program DEPG1 for extracting data and an initialization program INIT1. The means for transmitting data comprise the processor CPU1, a coding circuit CCT1 having an input coupled to a port of the processor, a mixer amplifier MD1 having a first input coupled to the output of the coding circuit CCT1 and a second input coupled to a voltage generator SG1, and a switch SW1 controlled by the processor, coupling the output of the amplifier MD1 to the electrode IE1. The receiving means comprise the processor CPU1 and a data acquisition chain 30. The acquisition chain 30 comprises a decoupling capacitor CC1, a low noise amplifier LNA1, a band-pass filter FM1 and an analog-digital converter ADC1 the output of which is coupled to a port of the processor CPU1. The amplifier LNA1 has an input coupled to the electrode IE1 through the decoupling capacitor CC1. The output of the amplifier is coupled to the input of the converter ADC1 through the band-pass filter FM1. The filter FM1 is centered on the transmitting frequency of the data signal SDT2 transmitted by the device D6.

During the phase PH1, when the device D5 is in receiving mode, the switch SW1 is open, the acquisition chain 30 receives the voltage V2(SDT2, BS) or a corresponding current and supplies the processor CPU1 with a filtered and digitized signal S(DT2, BS). By means of the program DEPG1, the processor demodulates the signal S(DT2, BS), extracts the data signal SDT2 from it, and then the data DT2 it comprises.

When the device D5 is in transmitting mode, the switch SW1 is closed, the processor supplies the coding circuit CCT1 with the data DT1, the circuit supplying the data signal SDT1. The amplifier MD1 modulates the amplitude of the voltage V1, supplied by the generator SG1, with the signal SDT1, and applies to the electrode IE1, via the switch SW1, the modulated voltage V1(SDT1). The initialization program INIT1 exchanges the data DT1, DT2 with the device D6 to determine the time at which the acquisition phase PH2 is triggered. During the phase PH2, the device D5 is in transmitting mode, the switch SW1 is closed, the coding circuit CCT1 is inactive, the amplifier MD1 receives the voltage V1 and applies it to the electrode IE1 without modulating its amplitude.

FIG. 10 shows an example of an embodiment of the device D6. The latter comprises a processor CPU2 coupled to a memory MEM2, means for transmitting data and a receiver circuit 100 configured to enable both the data DT1 sent by the device D5 to be received during the phase PH1, and the biological signal BS to be extracted during the phase PH2. The memory MEM2 comprises the program BEPG for extracting the signal BS, a program DEPG2 for extracting data, and an initialization program INIT2. It may also comprise the biological analysis program BAPG and the application program APG described above.

The means for transmitting data comprise the processor CPU2, a coding circuit CCT2 having an input coupled to a port of the processor, a mixer amplifier MD2 having a first input coupled to the output of the coding circuit CCT2 and a second input coupled to a voltage generator SG2, and a switch SW2 controlled by the processor, coupling the output of the amplifier MD2 to the electrode IE2.

The receiver circuit 100 comprises the processor CPU2 and a data and biological signal acquisition chain 40 the configuration of which is modified by the processor upon the switch from phase PH1 to phase PH2. The acquisition chain 40 comprises a decoupling capacitor CC2, a low noise amplifier LNA2, a band-pass filter FM2, and an analog-digital converter ADC2 the output of which is coupled to a port of the processor CPU2. The amplifier LNA2 has an input coupled to the electrode IE2 through the decoupling capacitor CC2. The output of the amplifier is coupled to the input of the converter ADC2 through the band-pass filter FM2. The filter FM2 is centered on the transmitting frequency of the data signal SDT1 transmitted by the device D6.

During the phase PH1, when the device D6 is in receiving mode, the switch SW2 is open, the acquisition chain 40 receives the voltage V2(SDT1, BS) or the current Id2 that it supplies to the processor CPU2 in the form of a filtered and digitized signal S(DT1, BS). By means of the program DEPG2, the processor demodulates the signal S(DT1, BS), extracts the data signal SDT1 from it, and then the data DT1. When the device D5 is in transmitting mode, the switch SW2 is closed, the processor supplies the coding circuit CCT2 with the data DT2, the circuit supplying the data signal SDT2. The amplifier MD2 modulates the amplitude of the voltage V1 supplied by the generator SG2 by means of the signal SDT2, and applies to the electrode IE2, via the switch SW2, the modulated voltage V1(SDT2). During the phase PH1, the initialization program INIT2 interacts with the program INIT1 of the device D5 by means of the data DT1, DT2, to determine the beginning of the phase PH2. According to one embodiment, the phase PH1 may also enable the device D6 to send the device D5 the biological signal BS or the biological parameter Bi it has extracted during a previous acquisition phase PH2.

At the beginning of the phase PH2, the acquisition chain 40 receives the voltage V2(BS) or the signal Id2 and supplies the processor CPU2 with the signal S(BS) in a digital form after removing the noise in the signal received. The processor demodulates and filters the signal S(BS) by means of the program BEPG, in the manner already described, to extract the biological signal BS. In one alternative, the device D6 comprises two distinct acquisition chains to respectively receive the data DT1 during the phase PH1 and the biological signal BS during the phase PH2. The device D6 may optionally comprise the program BAPG, to analyze the biological signal BS and extract from it a biological parameter Bi or an item of biological information Ii, and/or the biological application program APG, to exploit the biological signal BS, the biological parameter Bi or the biological information Ii.

FIG. 11 is a timing diagram showing the execution of the phases PH1, PH2. For the clarity of the diagram, the programs INIT1, INIT2, BAPG, APG are represented as distinct software entities of the devices D5, D6, considered here as physical layer means serving these software entities. Similarly, the body HB of the subject is considered a modulation means which transforms the signals V1 into signals V2 modulated by the biological signal BS. It can be seen that the programs INIT1, INIT2 interact via the data DT1, DT2 during the phase PH1. The program INIT1 supplies the device D5 with the data DT1, the device transmitting such data in the form of the modulated voltage V1(SDT1). The body HB transfers the signal V2(SDT1, BS) to the device D6 (or the signal Id(SDT1, BS), if reasoning in current), which extracts the data DT1 from this signal and supplies the program INIT2 with them. Similarly, the program INIT2 supplies the device D6 with the data DT2, the device transmitting them in the form of the modulated voltage V1(SDT2). The body HB transfers the signal V2(SDT2, BS) to the device D5, which extracts the data DT2 from this signal and supplies the program INIT1 with them. During the phase PH2, the device D5 transmits the signal V1, the body HB transfers the signal V2(BS) to the device D6, which extracts the biological signal BS. The analysis program BAPG extracts at least one biological parameter Bi or an item of biological information Ii from the biological signal. The application program APG may supply a result according to the biological signal BS, to the biological parameter Bi or to the biological information Ii, and implement applications such as heart monitoring, biometric identification, sleeping detection, etc.

The present invention is susceptible of various applications. In practice, at least one of the devices D3 and D4, or D5 and D6, may be installed in an object that a user often wears. For example, the device D4 or D6 may be installed in a watch, or in a cell phone MP, as shown on FIG. 12. The device D3 or D5 may be fixed and positioned at a given place, for example a table or a chair, close to the user. The biological signal BS can be detected as soon as the device D4 or D6 is close to the user, for example when the phone MP is held by the user or is in a pocket. Conversely, some embodiments may provide for the need for a voluntary movement by the user to trigger the acquisition of the biological signal BS. For example, if the device D3 or D5 is put on a table, provision may be made for the user, in order to trigger the acquisition of the biological signal BS, to have to place its hand on a zone of the device where the electrode IE1 is located, or to move it nearer to this zone.

In some embodiments, the programs BAPG and/or APG may be executed by the device D5 rather than by the device D6, the latter then transmitting to the device D5 the biological signal BS or the biological parameter Bi during the phase PH1.

Furthermore, the devices D3 to D5 described above are susceptible of various alternative embodiments. The filters FM, FM1, FM2 of the acquisition chains 20, 30, 40 may be digital filtering programs executed by the processor and applied to the digitized signal supplied by the converters ADC, ADC1, ADC2.

Finally, although it was stated above that the amplitude modulations of the IBAN signal that are representative of the biological signal BS are related to the heartbeat, subsequent studies could bring to light other cyclical factors impacting the electrical resistivity of the body, in particular the respiratory rhythm which influences the oxygenation of the blood and could also modulate the electrical resistivity of the body at a rate different from the heart rate, resulting in an additional modulation of the IBAN signal which can enable another biological signal to be extracted.

1. A receiving device with intracorporeal current comprising means for collecting, by capacitive coupling, an AC signal depending on a current that has passed through all or part of the body of a subject, and means for extracting data from the AC signal collected,
wherein it further comprises means for extracting from the AC signal a biological signal generated by the body of the subject and modulating the amplitude of the AC signal.

2. Device according to claim 1, configured to extract or extrapolate from the biological signal at least one biological parameter or one item of biological information.

3. Device according to claim 2, wherein the biological parameter is a parameter involved in transforming the heart signal into a biological signal.

4. Device according to claim 2, wherein the biological parameter consists of the variations in the subject's blood pressure.

5. Device according to claim 2, wherein the biological parameter the subject's heartbeat.

6. Device according to claim 2, wherein the biological information comprises at least one variation in the biological signal at a given time in the cardiac cycle.

7. Device according to claim 2, configured to develop a biometric identification datum of the subject from one or more biological parameters and/or one or more items of biological information.

8. Device according to claim 1, comprising means for transmitting data by applying to the body of the subject, by capacitive coupling, an AC signal modulated by a data signal.

9. A system with intracorporeal current comprising:
a transmitting device comprising means for applying to the body a subject, by capacitive coupling, a first AC signal, and means for transmitting data via the first AC signal, and
a receiving device according to claim 1, to collect, by capacitive coupling, a second AC signal, and to extract from the second AC signal data sent by the transmitting device,
in which the receiving device configured to:
during an initialization phase, exchange data with the transmitting device, and
during an acquisition phase, extract the biological signal from the second AC signal.

10. System according to claim 9, wherein at least one of the two devices is arranged in a portable object.

11. Method of acquiring a biological signal generated by the body of a subject, comprising steps of:
applying to the body of the subject a first electric AC signal, by means of a transmitting device comprising means for applying the first AC signal to the body of the subject by capacitive coupling, and means for transmitting data via the first AC signal,
collecting a second AC signal depending on a current that has passed through all or part of the body of the subject, by means of a receiving device comprising means for collecting, by capacitive coupling, the second AC signal, and means for extracting data from the AC signal collected, and
extracting the biological signal from the second AC signal, as signal modulating the amplitude of the second AC signal.

12. Method according to claim 11, wherein it comprises the steps of:
during an initialization phase, exchanging data with the transmitting device, by means of the receiving device, and
during an acquisition phase, extracting the biological signal from the second AC signal, by means of the receiving device, as signal modulating the amplitude of the second AC signal.

13. Method according to claim 11, comprising a step of extracting or extrapolating from the biological signal at least one biological parameter or one item of biological information.

14. Method according to claim 13, wherein the biological parameter is a parameter involved in transforming the heart signal into a biological signal.

15. Method according to claim 13, wherein the biological parameter consists of the variations in the subject's blood pressure or the subject's heartbeat.
